(19) United States
(12) Patent Application Publication
(10) Pub. No.: US 2016/0281077 A1
(43) Pub. Date: Sep. 29, 2016

(54) MEDIUM AND METHOD FOR CULTURING EUGLENA AND METHOD FOR REDUCING PHOTOINHIBITION IN EUGLENA

(71) Applicant: Euglena Co., Ltd., Tokyo (JP)

(72) Inventors: Akihiro OKAMOTO, Tokyo (JP); Kazuhito HASHIMOTO, Tokyo (JP); Yuki TOSHIMA, Tokyo (JP); Shuji NAKANISHI, Tokyo (JP); Iwao UEDA, Tokyo (JP); Nobuo AOKI, Tokyo (JP); Kengo SUZUKI, Kanagawa (JP)

(73) Assignee: Euglena Co., Ltd., Tokyo (JP)

(21) Appl. No.: 15/080,766

(22) Filed: Mar. 25, 2016

(30) Foreign Application Priority Data
Mar. 26, 2015 (JP) 2015-063801

(51) Int. Cl. C12N 13/00; C12N 1/12

(52) U.S. Cl. CPC C12N 13/00; C12N 1/12

(57) ABSTRACT

A medium for culturing Euglena includes a cell membrane-permeable electron mediator that includes a biocompatible moiety and a redox-active moiety. A method for culturing Euglena includes culturing Euglena in a medium including a cell membrane-permeable electron mediator that includes a biocompatible moiety and a redox-active moiety. The medium is placed in a fermenter system that includes a device for extracting electrons from the medium. A method for reducing photoinhibition in Euglena includes electrochemically culturing Euglena in a medium including a cell membrane-permeable electron mediator that includes a biocompatible moiety and a redox-active moiety. The medium is placed in a fermenter system that includes a device for extracting electrons from the medium.

### BACKGROUND OF THE INVENTION

1. Field of the Invention

The present invention relates to a medium and a method for culturing Euglena and a method for reducing photoinhibition in Euglena.

2. Description of the Related Art

Euglena is promising for use as fuels, food, animal feed, drugs, and the like.

When Euglena grows by fixing carbon dioxide by photosynthesis, Euglena produces oil and fat content, which can be used to produce a biofuel.

Unlike fossil fuels such as petroleum, biofuels do not raise concerns about depletion of resources. When fossil fuels are combusted as a fuel, carbon dioxide is generated. In contrast, biofuels are derived from plants and algae, which fix carbon dioxide as plants and algae are growing. Thus, emission of carbon dioxide is totally offset, which is believed to be effective in preventing global warming.

Increase in production of biofuels from edible parts of crops such as corn can cause decreased food supply and increased food prices. At present, Euglena is not used as a food source, thus production of biofuels from Euglena does not decrease food supply.

Euglena includes as many as 59 nutrients such as vitamins, minerals, amino acids, and unsaturated fatty acids, which represent the majority of the essential nutrients for humans. Previous studies have demonstrated the feasibility of using Euglena as supplements that provide a balanced combination of various nutrients and as food for nutritionally-deprived people in poor regions.

Euglena is a high protein source of high nutritive value and thus is expected to be used as feed for domestic animals and farmed fish.

Recent studies have demonstrated that materials derived from Euglena have various effects on humans, such as the effects of moisturizing skin, promoting wound healing, improving the barrier function of skin, and inhibiting allergic reactions, diabetes, and colon cancer. Drugs that take advantage of the above effects and use of Euglena as health food have been proposed.

As described above, Euglena exhibits great functional diversity and thus is promising for use as fuel, food, animal feed, and drugs. In recent years, as a result of their extensive research, researchers have achieved industrial production of Euglena and commercialization of products, such as health food, that include materials from Euglena.

On the other hand, Euglena is in the bottom of the food chain and is eaten by animals. It is more difficult to identify conditions, such as light, temperature, and agitation speed, for culturing Euglena, compared with other microorganisms. Thus, Euglena still cannot be produced in sufficiently high yields.

Especially, biofuels derived from Euglena is less cost-competitive than other fuels such as fossil fuels, which is one of the reasons why the biofuels from Euglena have not found widespread application. There are needs not only for a process for using Euglena to produce a fuel, but for increasing yields from a process for culturing Euglena and for lowering the manufacturing costs.

An outdoor fermenter system equipped with an agitator as a fermenter that can culture Euglena at a lower cost has been proposed (“Hitachi Plant Technologies Technical Journal No. 6”, [online], published on January 2012, Hitachi Plant Technologies, Ltd., [retrieved on Mar. 16, 2015], on the Internet (URL: www.hitachi.co.jp/products/inframoiety/product_site/randd/article/pdf/2012/gihou_201201.pdf; page 38).

The fermenter system is installed outdoors, which can result in reduced initial and operating costs and large-scale culture at a lower cost.

However, when Euglena is cultured in such outdoor fermenter, the Euglena loses significant weight during the night, which results in reduced growth rate and reduced productivity compared with indoor culture. It is believed that the low yields in outdoor culture can be attributed to temperature and sunlight energy.

### SUMMARY OF THE INVENTION

Through their extensive research to improve the yields obtained when Euglena is cultured outdoors, the inventors of the present invention have found that electrochemical culture of Euglena in a culture medium including a cell membrane-permeable electron mediator that includes a biocompatible moiety and a redox-active moiety can reduce photoinhibition, which is induced by excessive light conditions, and thus can inhibit the reduction of proliferation rate of Euglena cultured in an outdoor fermenter system and improve productivity of Euglena, thereby achieving the present invention.

Thus, the problems described above are solved through a medium for culturing Euglena, the medium including a cell membrane-permeable electron mediator that includes a biocompatible moiety and a redox-active moiety according to the present invention.

When the medium is used, Euglena may be electrochemically cultured. The mediator may be a polymer that includes 2-(methacryloyloxy)ethyl-2-(trimethylammonio)ethyl phosphate as the biocompatible moiety.

The mediator may include vinylferrocene as the redox-active moiety and may be poly(MPC-co-VF) represented by the following General Formula (I):

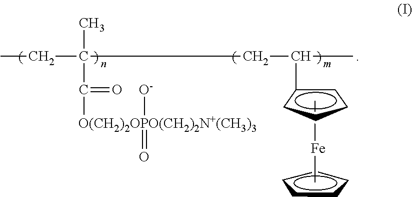

The problems described above are solved through a method for culturing Euglena according to the present invention, the method including culturing Euglena in a medium including a cell membrane-permeable electron mediator that includes a biocompatible moiety and a redox-active moiety medium. The medium is placed in a fermenter system that includes a device for extracting electrons from the medium.

The device for extracting electrons may be an electrode. In the method, Euglena may be electrochemically cultured. The mediator may be a polymer that includes 2-(methacryloyloxy)ethyl-2-(trimethylammonio)ethyl phosphate as the biocompatible moiety.

The mediator may include vinylferrocene as the redox-active moiety and may be poly(MPC-co-VF) represented by the following General Formula (I):

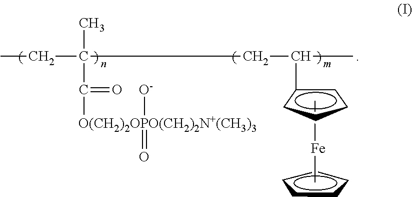

The problems described above are solved through a method for reducing photoinhibition in Euglena according to the present invention, the method including electrochemically culturing Euglena in a medium including a cell membrane-permeable electron mediator that includes a biocompatible moiety and a redox-active moiety. The medium is placed in a fermenter system that includes an electrode.

The mediator may be a polymer that includes 2-(methacryloyloxy)ethyl-2-(trimethylammonio)ethyl phosphate as the biocompatible moiety.

The mediator may also include vinylferrocene as the redox-active moiety and may be poly(MPC-co-VF) represented by the following General Formula (I):

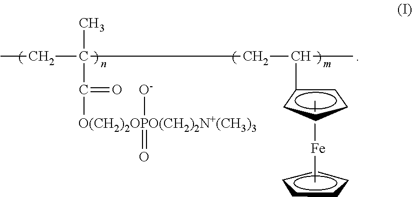

As used herein, the term photoinhibition means that excessive light conditions induce damage to photosystem II (PSII) of photosynthetic organisms, which results in decrease in photosynthetic activity and photosynthetic efficiency.

As used herein, the term strong light is used interchangeably with excessive light conditions and refers to light having an intensity above the photosaturation point of a photosynthetic organism.

The term photosaturation point refers to the intensity of light beyond which further increases in light intensity will not lead to an increase in rate of photosynthesis of a photosynthetic organism. Different photosynthetic organisms have different photosaturation points.

Some photosynthetic microorganisms including microalgae such as Euglena exhibit a proliferation rate that increases with increase in light intensity when the microorganisms are exposed to light having an intensity lower than the intensity at the photosaturation point and exhibit a reduced proliferation rate when the microorganisms are exposed to light having an intensity higher than the intensity at the photosaturation point.

When photosynthetic organisms are exposed to light having a weak intensity that is lower than the intensity at the photosaturation point, NADPH produced by light reactions acts as a source of reducing power and are consumed in the Calvin cycle, which is a metabolic cycle that fixes and reduces CO₂. Under strong light, the rate of production of the source of reducing power by light reactions increases and then exceeds the rate of the CO₂ fixation reaction in the Calvin cycle. As illustrated in FIG. 7, the excessive reducing power is transported to oxygen, producing reactive oxygen species such as singlet oxygen (¹O₂), superoxide anion (O₂⁻), and hydrogen peroxide (H₂O₂) from a downstream reaction (Foyer et al., Physiol. Plant. 92:696-717 (1994)). These reactive oxygen species, which are highly reactive, damage proteins, lipids, and DNA and leads to photoinhibition, which inhibits growth and proliferation of photosynthetic organisms.

The medium and the method for culturing Euglena and the method for reducing photoinhibition in Euglena according to the present invention can eliminate excessive reducing power induced by strong light and can reduce photoinhibition, because the cell membrane-permeable electron mediator that includes a biocompatible moiety and a redox-active moiety transports excessive electrons produced in Euglena cell from the photosynthetic electron transport system to an extracellular electrode through extracellular electron transfer.

Thus, the present invention can reduce variation in the yield of Euglena due to the variation in solar intensity when Euglena is cultured outdoors.

The cell membrane-permeable electron mediator according to the present invention includes a biocompatible moiety in addition to a redox-active moiety included in conventional electron mediators and thus allows the extracellular electron transfer from the inside of Euglena cell to an extracellular electrode without breaking the cell membranes of Euglena.

Because the medium and the method for culturing Euglena and the method for reducing photoinhibition in Euglena according to the present invention can reduce photoinhibition during culture of Euglena, the prevent invention can also improve productivity when Euglena is produced in large quantities by using an outdoor fermenter system with a low installation cost and can increase the yield of a useful material for use as biofuels, food, or pharmaceutical materials derived from Euglena.

The excessive reducing power transferred from the cell by the cell membrane-permeable mediator can also be used.

### BRIEF DESCRIPTION OF THE DRAWINGS

FIG. 1 illustrates a fermenter system for carrying out a method for culturing Euglena and a method for reducing photoinhibition in Euglena according to an embodiment of the present invention.

FIG. 2 is a graph of the dependence of photocurrent formed by extracellular transfer of electrons in Euglena on intensity of irradiating light.

FIG. 3 is a graph of the proliferation of Euglena cultured in a medium supplemented with or without poly(MPC-co-VF) (hereinafter referred to as “PMF”) under strong light.

FIG. 4 is a graph of the proliferation of Euglena cultured in a medium supplemented with or without PMF under weak light.

FIG. 5A is a graph of the proliferation of Euglena cultured in a medium supplemented with or without an antioxidant under strong light as a reference study for confirming the effect of electrochemical culture of Euglena using PMF.

FIG. 5B is a graph of the proliferation of Euglena cultured in a medium supplemented with or without an antioxidant under weak light as a reference study for confirming the effect of electrochemical culture of Euglena using PMF.

FIG. 6 is a graph of the photocurrent when electron transport inhibitor DBMIB is added after Euglena is added as a reference test for confirming the effect of electrochemical culture of Euglena using PMF.

FIG. 7 schematically illustrates the mechanism of photoinhibition.

### DETAILED DESCRIPTION OF THE INVENTION

The present invention relates to a medium and a method for culturing Euglena and a method for reducing photoinhibition in Euglena. Now, the medium and the method for culturing Euglena and the method for reducing photoinhibition in Euglena according to the present invention will be described in detail.

Unless otherwise defined herein, all technical and scientific terms used herein have the same meaning as commonly understood by one of ordinary skill in the art to which the present invention belongs. Although any methods and materials similar or equivalent to those described herein can be used in the practice or testing of the present invention, the preferred methods and materials are now described.

Euglena

The term “Euglena” in the present invention includes all of the plants that belong to genus Euglena according to the zoological and botanical classification, varieties thereof, and variants thereof, and plants including a component that is effective in inhibiting α-glucosidase activity.

In zoology, the microorganisms of the genus Euglena belong to the suborder Euglenoidina of the order Euglenida of the class Phytomastigophorea of the class Mastigophorea of the phylum Protozoa. In botany, the microorganisms of the genus Euglena belong to the order Euglenales of the class Euglenophyceae of the phylum Euglenophyta.

Specific examples of the microorganisms of the genus Euglena include Euglena acus, Euglena caudata, Euglena chadefaudii, Euglena deses, Euglena gracilis, Euglena granulata, Euglena intermedia, Euglena mutabilis, Euglena oxyuris, Euglena proxima, Euglena spirogyra, Euglena viridis, Euglena vermiformis, Euglena intermedia, and Euglena piride.

Among them, Euglena gracilis, which is widely used in studies, is particularly preferred. Examples of Euglena gracilis include the strain Euglena gracilis Z.

Additionally, species such as Euglena gracilis Klebs and Euglena gracilis var. bacillaris, the strain SM-ZK, which is a chloroplast deficient mutant derived from the strain Euglena gracilis Z, var. bacillaris, and genetic mutant strains such as chloroplast mutants thereof may be used. Other Euglena such as Astaia longa may also be used.

Euglena generally live in fresh water such as pools and ponds, and thus Euglena may be isolated from the fresh water. Alternatively, any previously-isolated Euglena may be used.

In the present invention, Euglena includes all mutant strains. The mutant strains encompass mutants produced through genetic techniques such as recombination, transduction, and transformation.

Medium for Culturing Euglena

A medium for culturing Euglena according to an embodiment of the present invention is a nutrient medium that includes nutrients required for growth of Euglena and that is supplemented with a cell membrane-permeable electron mediator that includes a biocompatible moiety and a redox-active moiety.

The nutrients required for growth of Euglena include, for example, nutrient salts such as nitrogen sources, phosphorus sources, and minerals.

The nutrient medium that includes the nutrients required for growth of Euglena is a known nutrient medium commonly used for culture of Euglena. For example, it is possible to use modified Cramer-Myers medium (1.0 g/L of (NH₄)₂HPO₄, 1.0 g/L of KH₂PO₄, 0.2 g/L of MgSO₄.7H₂O, 0.02 g/L of CaCl₂.2H₂O, 3 mg/L of FeSO₄.7H₂O, 1.8 mg/L of MnCl₂.4H₂O, 1.5 mg/L of CoSO₄.7H₂O, 0.4 mg/L of ZnSO₄.7H₂O, 0.2 mg/L of Na₂MoO₄.2H₂O, 0.02 mg/L of CuSO₄.5H₂O, 0.1 mg/L of thiamine hydrochloride (vitamin B₁), cyanocobalamin (vitamin B₁₂), (pH 3.5)). It is noted that (NH₄)₂HPO₄ may be displaced by (NH₄)₂SO₄ or NH_{3aq}.

The nutrient medium may also be known Hutner's medium or Koren-Hutner's medium (hereinafter referred to as “KH medium”) prepared as described in “Euglena—Physiology and Biochemistry” (Kitaoka, S (ed.), Gakkai Shuppan Center, K.K.), or AY medium, which is an autotrophic medium prepared by removing heterotrophic nutrients such as glucose, malic acid, and amino acids from Koren-Hutner's medium, which is commonly used as a heterotrophic medium for Euglena.

The nutrient medium preferably has a pH of 2 or higher, more preferably 3 or higher, and still more preferably 3.5 or higher, and preferably 7 or less, more preferably 6.5 or less, and still more preferably 4.5 or less. In a culture medium having an acidic pH, photosynthetic microorganisms can grow better than other microorganisms, which can prevent contamination.

The cell membrane-permeable electron mediator according to an embodiment of the present invention is a phospholipid polymer that includes a biocompatible moiety and a redox-active moiety.

The biocompatible moiety is highly biocompatible because of its high resistance to protein adsorption and preferably includes 2-(methacryloyloxy)ethyl-2-(trimethylammonio)ethyl phosphate (hereinafter referred to as “MPC”).

The redox-active moiety includes a redox mediator and preferably vinylferrocene.

The redox-active moiety may include another redox mediator such as a compound having a quinone moiety, a compound having a ferrocene moiety, or another compound. Examples of the compound having a quinone moiety include benzoquinones and compounds having a naphthoquinone moiety or an anthraquinone moiety. In addition to vinylferrocene, examples of the compound having a ferrocene moiety include dimethylaminomethyl ferrocene, 1,1′-bis(diphenylphosphino)ferrocene, dimethyl ferrocene, and ferrocene monocarboxylic acid. Examples of the another compound include iron (Fe) complexes, compounds having a nicotinamide moiety, compounds having a riboflavin moiety, and compounds having a nucleotide phosphate moiety.

The General Formula (I) represents an exemplary biocompatible redox phosphate polymer, PMF (poly(MPC-co-VF)), which includes MPC as the biocompatible moiety and vinylferrocene as the redox-active moiety:

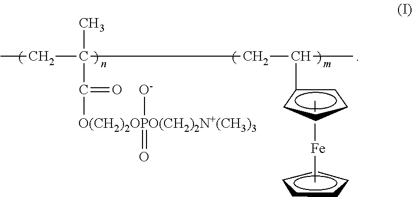

In the embodiment, PMF is prepared by free radical polymerization of MPC with vinylferrocene in approximately the same ratio, and the ratio of MPC and the ratio of vinylferrocene are respectively about 52 mol % and about 48 mol %. The PMF has a weight average molecular weight (M_{w}) of 6.6 kDa and a polydispersity (M_{w}/Mₙ) of 1.7.

In cyclic voltammetry of PMF in PBS, a reversible redox wave was observed with a midpoint potential of +0.5 V vs. SHE. Thus, PMF can accept electrons from redox active species in cells, such as nicotinamide adenine dinucleotide (E₀′: −0.3 V) and ubiquinone (E₀′: +0.1 V).

In the embodiment, the cell membrane-permeable electron mediator includes a biocompatible moiety and thus allows transportation of electrons from microorganisms such as microalgae to an extracellular electrode through the cell membranes without breaking cell membranes and biomolecules in a long period of electrochemical culture.

Fermenter System

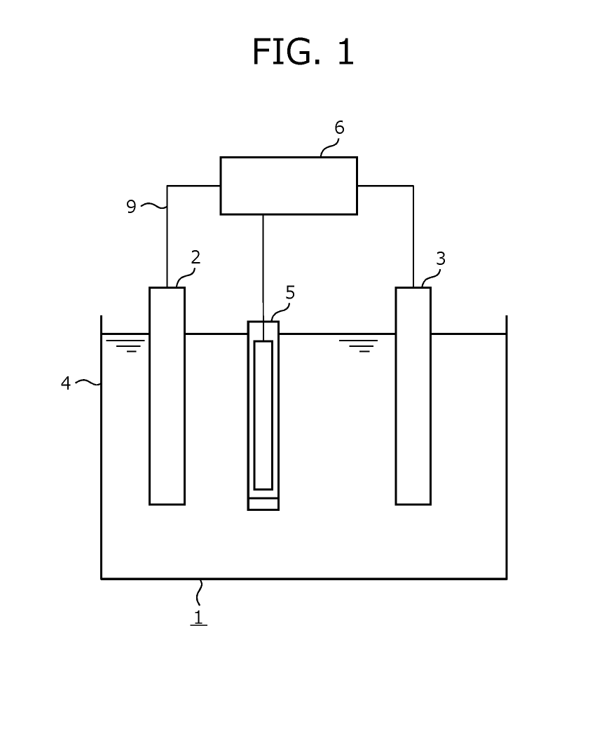

In an embodiment of the present invention, a fermenter system 1 as illustrated in FIG. 1 is used to electrochemically culture Euglena.

The fermenter system 1 includes a fermenter tank 4 for receiving and storing a culture medium according to an embodiment of the present invention, a first electrode 2 as a sample electrode (working electrode), a second electrode 3 as a counter electrode, a standard electrode 5 as a reference electrode, and a potentiostat 6 for controlling a potential on the first electrode 2. The first electrode 2, the second electrode 3, and the standard electrode 5 represent a device for extracting electrons from the medium.

In the embodiment, the fermenter tank 4 includes an open pond fermenter tank that is installed outdoors. The fermenter tank 4 is fitted with an agitator that includes a mechanism such as a paddle wheel or airlift. Preferred examples of the fermenter tank include, but are not limited to, raceway fermenter tanks, which are shaped like a racing circuit and provide a flow-through system.

The first electrode 2 and the second electrode 3 may be formed of any suitable material. For example, the first electrode 2 may be an indium tin oxide (no) electrode or a glassy carbon (GC) electrode, and the second electrode 3 may be a platinum electrode.

Examples of the standard electrode 5 include, but are not limited to, silver/silver chloride electrodes.

The potentiostat 6 controls a potential on the first electrode 2 to a predetermined potential relative to the standard electrode 5. The control may be performed by a different configuration from the configuration in the embodiment as long as the potentiostat can control a potential on the first electrode 2. The fermenter system does not need to include the standard electrode 5 and may include a voltage applicator (not shown) in place of the potentiostat 6. The voltage applicator may be configured to apply a predetermined voltage between the first electrode 2 and the second electrode 3

Electrochemical Culture of Euglena

A method for culturing Euglena and a method for reducing photoinhibition in Euglena according to an embodiment of the invention include electrochemically culturing Euglena in the fermenter system 1 as illustrated in FIG. 1, the fermenter system 1 including a culture medium according to an embodiment of the present invention.

For the electrochemical culture, first, a culture medium according to an embodiment of the present invention and Euglena are placed into the fermenter system 1 illustrated in FIG. 1. Then, while maintaining a pH of from 2 to 4 and a depth of from 20 to 30 cm, the culture medium is circulated using a paddle or airlift system to culture the Euglena. During the culture, the medium may be gassed with 5-15% carbon dioxide. The culture time should include the daytime, when the sun is shining.

In the embodiment, Euglena is cultured using the medium and the method for culturing Euglena or the method for reducing photoinhibition in Euglena according to the invention, although other photosynthetic microorganisms such as other eukaryotic microalgae may be cultured using the medium and the method for culturing Euglena or the method for reducing photoinhibition in Euglena according to the present invention.

### SPECIFIC EXAMPLES

Now, the present invention will be specifically described with reference to specific examples, although the present invention is not limited to the examples.

Preculture of Euglena

Euglena gracilis Z was used as a Euglena sample. First, the Euglena was precultured in the following manner.

29 ml of an autoclave-sterilized CM medium was placed into an autoclave-sterilized vial. Then, 1 ml of the nutrient medium including the Euglena was added. Then, the nutrient medium was incubated in an incubator at 30° C. under 5000 lux light (from 20 W fluorescent tube BIOLUX A from NEC Corp.) with air bubbling for about a week.

The CM medium had a composition of 1 g/L of (NH₄)₂HPO₄, 1 g/L of KH₂PO₄, 0.2 g/L of MgSO₄.7H₂O, 0.02 g/L of CaCl₂.2H₂O, 3.0 mg/L of Fe₂(SO₄)₃.7H₂O, 1.8 mg/L of MnCl₂.4H₂O, 1.5 mg/L of CoSO₄.7H₂O, 0.4 mg/L of ZnSO₄.7H₂O, 0.2 mg/L of Na₂MoO₄.2H₂O, 0.02 mg/L of CuSO₄5H₂O, 0.1 mg/L of thiamine hydrochloride (vitamin B₁), and 0.001 mg/L of cyanocobalamin (vitamin B₁₂). The CM medium was prepared with reference to a publication by Marian et al., (1952). The pH was adjusted to about 3.5 with concentrated sulfuric acid.

Studies: Measurement of Photocurrent to Confirm Transportation of Electrons from Euglena to Extracellular Electrode

If an extracellular electron transfer pathway exists between the photosynthetic electron transport system in Euglena cell and the extracellular electrode, electrons produced through exposure of Euglena to light are transported to the electrode, which can be observed as a rise in current. In the examples, existence of the extracellular electron transfer pathway was confirmed by measuring photocurrent, which is an electric current produced by a photoelectric effect.

Study 1: Change in Photocurrent in Response to Change in Light Intensity

The CM medium having a PMF concentration of 5 g/L was placed into a three-electrode electrochemical cell that included an indium tin oxide (ITO) electrode as a working electrode, a platinum electrode as a counter electrode, a silver/silver chloride (Ag/AgCl) electrode as a reference electrode, and a 4 mL fermenter tank. The PMF had a redox potential of +0.3 V vs. Ag/AgCl.

To the medium, the precultured Euglena was added at an initial density (OD800) of 3. Then, the electrochemical cell was placed into a shielded enclosure.

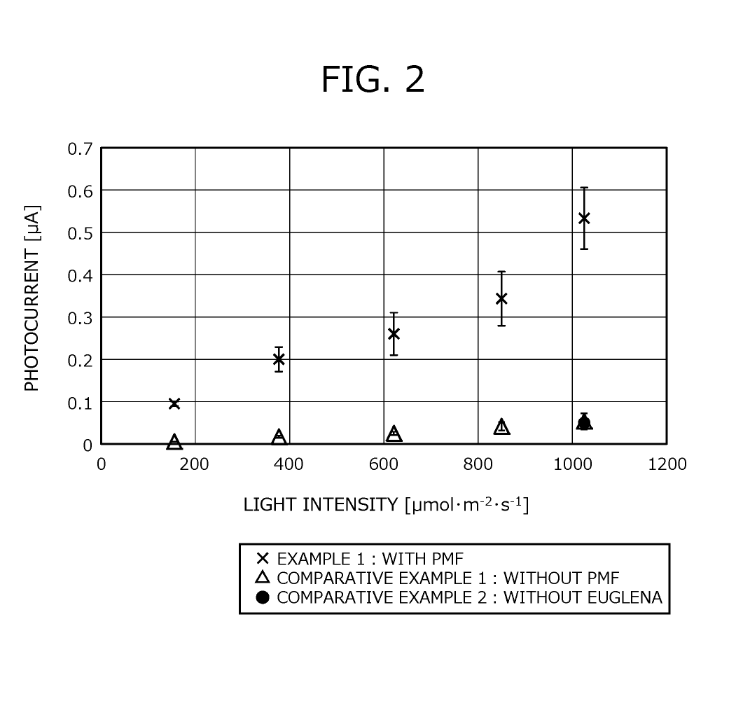

The Euglena in the medium was exposed to light at a single wavelength of 680 nm at five different light intensities in a range of from 155 to 1025 μmol·m-2·s-1 as illustrated in FIG. 2 for 30 seconds through the ITO transparent electrode as the working electrode to measure the rise in current as the photocurrent (Example 1). The potential was set at +0.6 V vs SHE.

A similar study was performed using a medium that included no PMF (Comparative Example 1). A study was also performed using the CM medium that had a PMF concentration of 5 g/L and that included no Euglena (Comparative Example 2).

The results are illustrated in the graph of the dependence of photocurrent formed by extracellular transfer of electrons on intensity of irradiating light of FIG. 2. In the Example 1, as illustrated in FIG. 2, the photocurrent rose in response to an increase in the intensity of irradiating light. In the Comparative Example 1 without PMF and the Comparative Example 2 without Euglena, the current did not rise in response to an increase in the intensity of irradiating light. The results show that PMF transferred electrons through the extra cellular electron transfer, thereby causing extraction of electrons from the photosynthetic electron transport system in Euglena cell to the extracellular electrode.

In the Example 1, which used the medium and the electrochemical culture according to the present invention, the electrons produced through exposure of the Euglena to light were transferred to the electrode, which was observed as a rise in current. This indicates that the cell membrane-permeable mediator acted as an extracellular electron transfer pathway between the photosynthetic electron transport system in the Euglena cell and the extracellular electrode. In the extracellular electron transfer pathway mechanism, the electrode acted as a device for extracting electrons from the medium.

In the medium and the electrochemical culture in the Example 1, excessive electrons in the Euglena cell were extracted to the extracellular electrode through the extracellular electron transfer pathway between the photosynthetic electron transport system in the Euglena cell and the electrode.

Study 2: Effect of Reducing Proliferative Inhibition

In the example, proliferation of Euglena electrochemically cultured under strong light, Euglena electrochemically cultured under weak light, and Euglena non-electrochemically cultured was compared.

The CM medium having a PMF concentration of 0.5 g/L was placed into the same three-electrode electrochemical cell as the Study 1.

To the medium, the precultured Euglena was added at an initial density (OD800) of 0.05 and electrochemically cultured under strong light having an intensity of 5000 lux, which is about one tenth of the maximum intensity of sunlight. After 0, 24, 48, 76, 96, and 148 hours from the start of the culture, the cell density was determined (Example 2). The voltage applied was +0.6 V vs. SHE.

A similar study was performed on Euglena non-electrochemically cultured in the medium that included no PMF (Comparative Example 3).

A similar study was also performed on Euglena electrochemically cultured in the medium that included PMF (Comparative Example 4) and Euglena electrochemically cultured in the medium that included no PMF (Comparative Example 5) under weak light having an intensity of 1000 lux, which is about one fiftieth of the maximum intensity of sunlight.

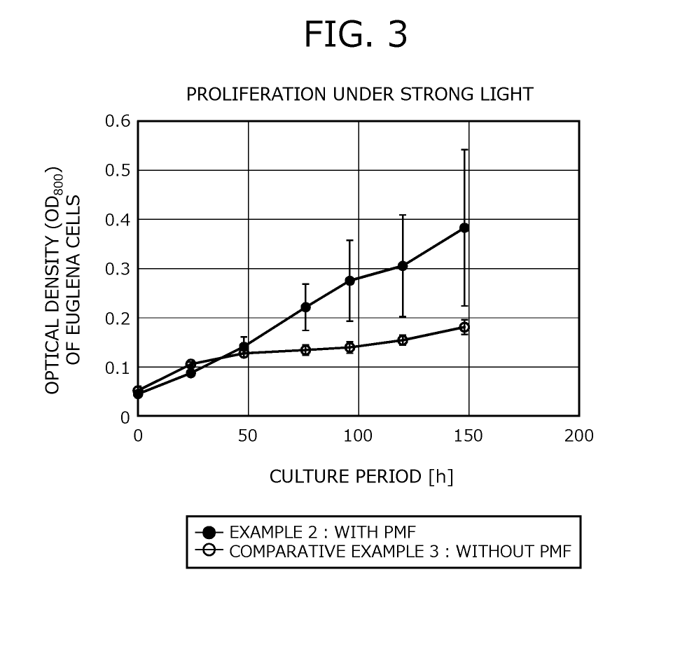

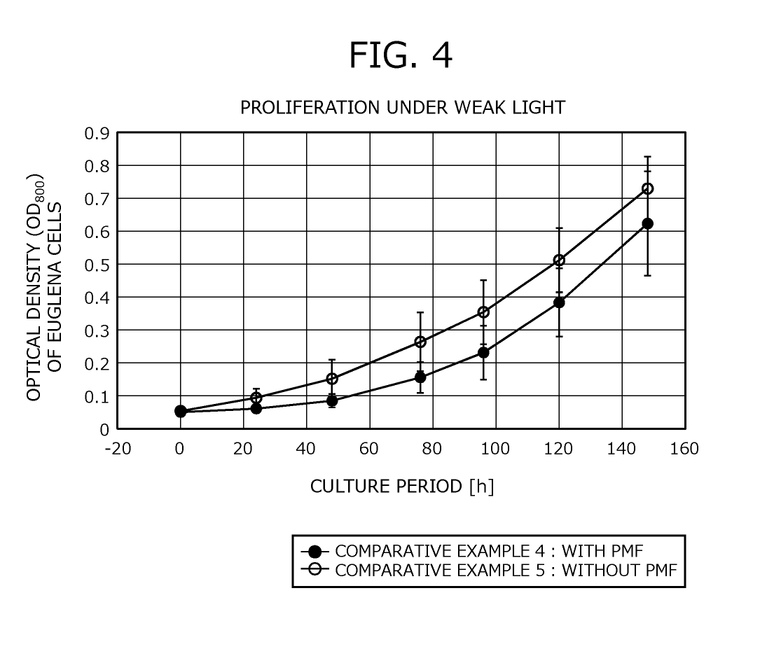

The results are illustrated in the graphs of the proliferation of Euglena under strong light (FIG. 3) and under weak light (FIG. 4).

As illustrated in FIG. 3, the Euglena that were electrochemically cultured at +0.6 V vs. SHE in the medium that included PMF under strong light in the Example 2 exhibited a significantly higher proliferation rate compared with the Euglena that is non-electrochemically cultured in the medium that included no PMF under strong light in the Comparative Example 3. As illustrated in FIG. 4, the Euglena that is electrochemically cultured at +0.6 V vs. SHE in the medium that included PMF under weak light in the Comparative Example 4 exhibited a lower proliferation rate compared with the Euglena that were non-electrochemically cultured in the medium that included no PMF in the Comparative Example 5.

The results reveal that electrochemical culture in the medium that includes PMF under strong light facilitates the proliferation of Euglena. The results also reveal that addition of PMF to a medium is not effective in facilitating the proliferation when Euglena is cultured under weak light and that addition of PMF is effective only when Euglena is cultured under strong light.

Reference Study: Effect of Antioxidant in Facilitating Proliferation under Strong Light and Weak Light

To examine the mechanism of action of the present invention, reference study was performed using the medium supplemented with ascorbic acid, which is an antioxidant.

In the study, proliferation of Euglena that were cultured in the medium supplemented with or without 0.5 mM of ascorbic acid in place of PMF used in the Study 2 under strong light or under weak light was compared. The study was performed in the same manner as in the Study 2 except that 0.5 mM of ascorbic acid was added in place of PMF.

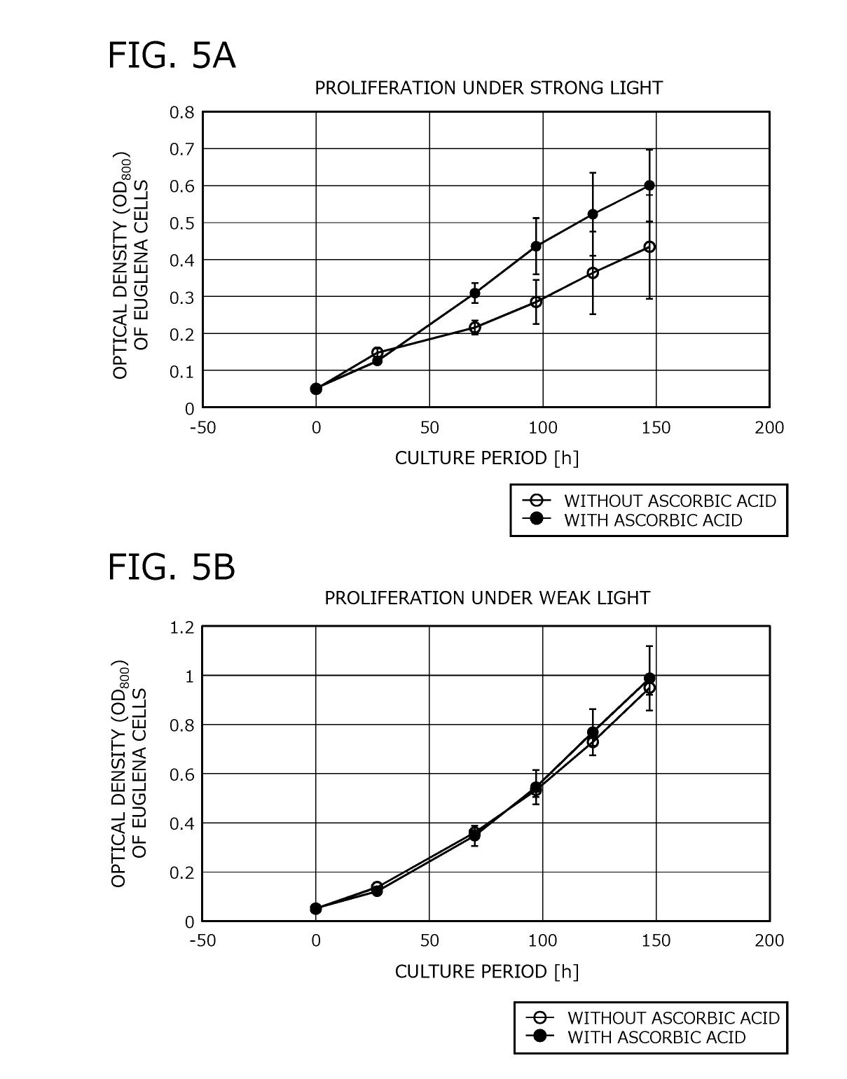

The results are illustrated in FIG. 5. As illustrated in FIG. 5, culture in the medium supplemented with ascorbic acid under strong light was effective in facilitating the proliferation of Euglena, while the culture under weak light was not effective in facilitating the proliferation.

The results illustrated in FIGS. 3-5 show that as with ascorbic acid, which is an antioxidant, PMF was effective in facilitating the proliferation of Euglena in culture under strong light. As with ascorbic acid, which is an antioxidant, PMF was not effective in facilitating the proliferation of Euglena in culture under weak light.

Study 3: Effect of Addition of DBMIB, which is Electron Transport Inhibitor

To determine whether the effect of the medium supplemented with PMF in facilitating the proliferation of Euglena in electrochemical culture as shown by the Studies 1 and 2 is attributed to extracellular transfer of electrons from Euglena, DBMIB (2,5-dibromo-3-methyl-6-isopropyl-p-benzoquinone), which is an electron transport inhibitor, was added to the culture system that included PMF, and a change in photocurrent was measured.

DBMIB binds to the plastoquinone-binding site of the cytochrome b6f and prevents the oxidation of reduced plastoquinone thereby inhibiting electron transport. DBMIB inhibits the transfer of electrons from plastoquinone to the photosystem I and thus is used to measure the activity of the photosystem II.

In the Study 3, the CM medium having a PMF concentration of 3 g/L was placed into the same three-electrode electrochemical cell as the Study 1. Then, the electrochemical cell was placed into a shielded enclosure to measure the photocurrent.

After about 21 minutes from the start of the measurement, the precultured Euglena was added at an initial density (OD800) of 0.5. After about 37 minutes from the start of the measurement, DBMIB was added at a concentration of 100 μM to the medium.

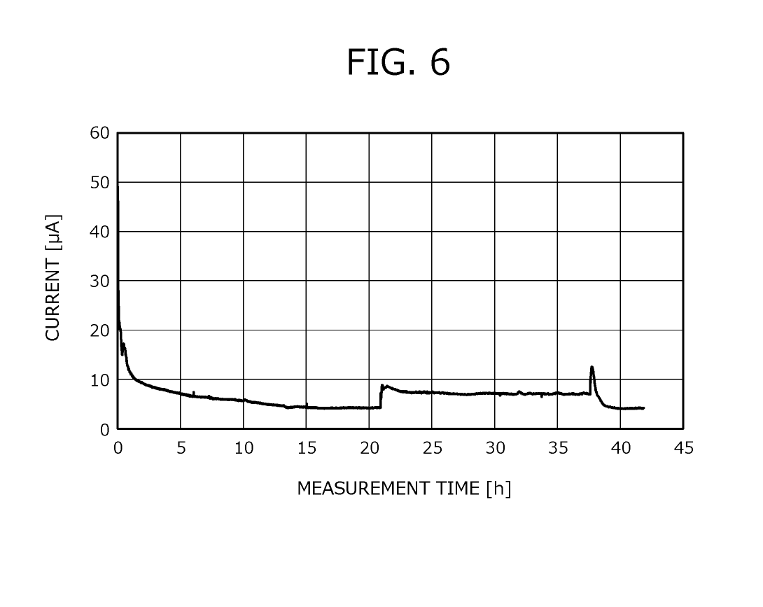

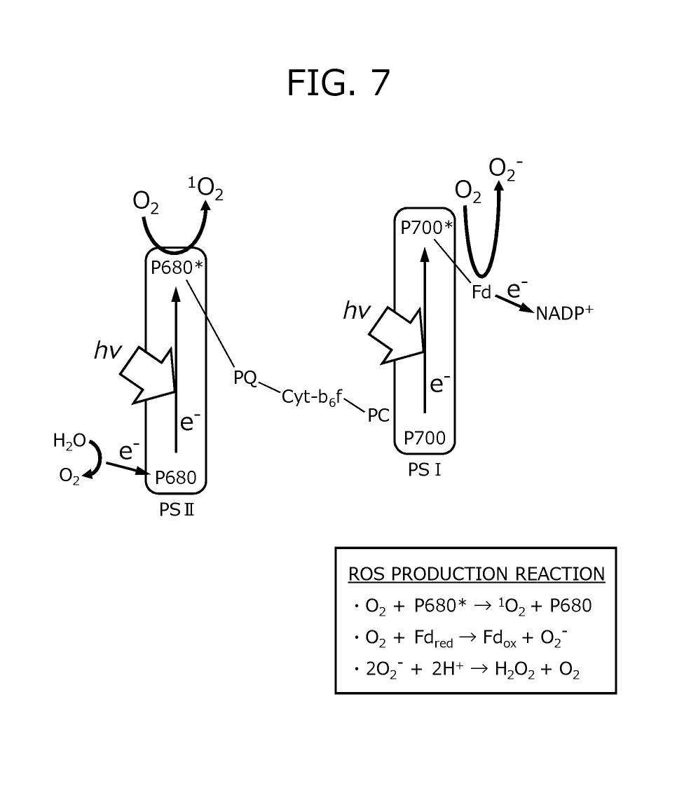

The results are illustrated in FIG. 6. As illustrated in FIG. 6, when Euglena was added after about 21 minutes from the start of the measurement, the current rose from about 4 μA to about 8 μA. When DBMIB was added after about 37 minutes from the start of the measurement, the current returned to about 4 μA.

The decrease in photocurrent due to the addition of DBMIB shows that the extracellular electron transfer pathway existed before the addition of DBMIB.

What is claimed is:

1. A medium for culturing Euglena, the medium comprising a cell membrane-permeable electron mediator that comprises a biocompatible moiety and a redox-active moiety.

2. The medium for culturing Euglena according to claim 1,
wherein the Euglena is electrochemically cultured, and
wherein the mediator is a polymer that comprises 2-(methacryloyloxy)ethyl-2-(trimethylammonio)ethyl phosphate as the biocompatible moiety.

3. The medium for culturing Euglena according to claim 2, wherein the mediator comprises vinylferrocene as the redox-active moiety and is poly(MPC-co-VF) represented by the following General Formula (I):

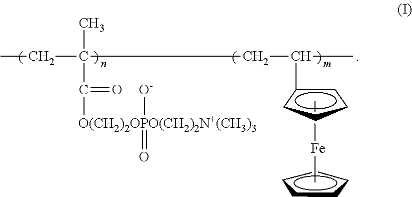

4. A method for culturing Euglena, the method comprising culturing Euglena in medium comprising a cell membrane-permeable electron mediator that comprises a biocompatible moiety and a redox-active moiety, wherein the medium is placed in a fermenter system that comprises a device for extracting electrons from the medium.

5. The method for culturing Euglena according to claim 4,
wherein the device for extracting electrons is an electrode,
wherein the Euglena is electrochemically cultured, and
wherein the mediator is a polymer that comprise 2-(methacryloyloxy)ethyl-2-(trimethylammonio)ethyl phosphate as the biocompatible moiety.

6. The method for culturing Euglena according to claim 5, wherein the mediator comprises vinylferrocene as the redox-active moiety and is poly(MPC-co-VF) represented by the following General Formula (I):

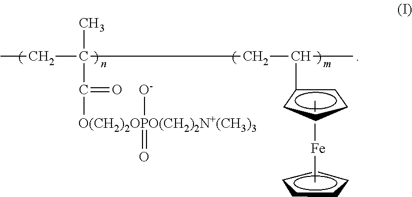

7. A method for reducing photoinhibition in Euglena, the method comprising electrochemically culturing Euglena in a medium comprising a cell membrane-permeable electron mediator that comprises a biocompatible moiety and a redox-active moiety, wherein the medium is placed in a fermenter system that comprises an electrode.

8. The method for reducing photoinhibition in Euglena according to claim 7, wherein the mediator is a polymer that comprises 2-(methacryloyloxy)ethyl-2-(trimethylammonio)ethyl phosphate as the biocompatible moiety.

9. The method for reducing photoinhibition in Euglena according to claim 8, wherein the mediator comprises vinylferrocene as the redox-active moiety and is poly(MPC-co-VF) represented by the following General Formula (I): [chemical structure]
